(19) United States
(12) Patent Application Publication
(10) Pub. No.: US 2019/0374220 A1
(43) Pub. Date: Dec. 12, 2019

(54) APPARATUS AND METHODS FOR APPROXIMATING AND SECURING TISSUE

(71) Applicant: QuickStitch Surgical, Inc., Berkeley, CA (US)

(72) Inventors: Rachel FISCHELL, Los Angeles, CA (US); Gil LAROYA, Santa Clara, CA (US); Duncan HUGHES, Durham, CA (US); Piyani GANDHI, New Albany, IN (US); Orvil COLLART, Brussels, CA (US)

(73) Assignee: QuickStitch Surgical, Inc., Berkeley, CA (US)

(21) Appl. No.: 16/376,212

(22) Filed: Apr. 5, 2019

Related U.S. Application Data
(60) Provisional application No. 62/653,217, filed on Apr. 5, 2018.

(51) Int. Cl. A61B 17/04; A61B 17/06

(52) U.S. Cl. CPC A61B 17/0482; A61B 17/0491; A61B 2017/303; A61B 17/06114; A61B 17/06109

(57) ABSTRACT

Disclosed is a device that uses suture to close various layers of tissue quickly and precisely. The device and methods enhance the efficiency, consistency and cost-effectiveness of traditional hand-suturing without compromising cosmetic or wound healing outcomes. Adjustable instruments on either side of the device are applied to opposing sides of the desired layer of tissue to approximate the edges of the tissue to be closed. The user may control the delivery of a single stitch or a series of stitches at an adjustable interval to the desired layer of tissue. An instrument may perform the stitch or series of stitches as the operator guides the device along the tissue opening. Once the device has delivered the appropriate stitch(es), the user may remove excess suture material and release the tissue from the instruments on either side of the incision and the process may be repeated as necessary to close a wound.

### CROSS-REFERENCE TO RELATED APPLICATIONS

This application claims the benefit of priority to U.S. Prov. 62/653,217 filed Apr. 5, 2018, which is incorporated herein by reference in its entirety.

### FIELD OF THE INVENTION

The present invention relates generally to surgical instruments utilized intraoperatively for wound or incision closure. More specifically, the present invention allows for the utilization of a device to automate aspects of traditional suturing methods more quickly and precisely than if done manually using traditional techniques.

### BACKGROUND OF THE INVENTION

To enable biological healing of a tissue opening, the tissue surfaces on either side of the wound must be in close proximity. Under conditions where a physician believes that the two sides of the tissue will not naturally remain in close enough proximity to promote healing, he/she will forcibly unite the sides of the opening. Although the use of metal and bioabsorbable surgical staplers as well as external sutureless closure devices is becoming increasingly popular for closure of external layers, traditional suturing remains the most prevalent method of effectuating closure of wound openings in a variety of biological layers, including, but not limited to organs, muscles, fascia, subcutaneous fat, and dermal layers.

Many invasive surgical procedures require a surgeon to make an incision through multiple biological layers. At the end of the procedure, each of those layers must be sutured back together edge to edge, one layer at a time. Traditionally, the clinician begins by closing the deepest interior layer and works toward the most superficial layer.

Current methods of closing tissue using suture involve manually inserting a length of material that is attached to a tissue-piercing device, such as a needle, through the opposing sides of an opening. The suture is then pulled tight causing the opposing sides of the tissue to come into close physical proximity. The suture is held tight by the tying of a knot or some other locking mechanism. For procedures requiring particularly long incisions through multiple biological layers, this process can be time consuming. Time spent suturing warrants excessive time spent under anesthesia, increasing the risk to the patient. A longer procedure time also increases the cost of the procedure. Furthermore, in cases where the closure is done poorly or takes too long, complications such as excessive bleeding, infection, seroma, incisional hernia, wound dehiscence (wound rupture), tissue devascularization, necrosis (tissue death), or undesirable cosmetic results can occur. Clinicians are as consistent as possible when suturing but may not always be precise, especially if rushed.

### SUMMARY OF THE INVENTION

Disclosed are devices and methods that can use suture to close various layers of tissue quickly and precisely. These devices and methods enhance the efficiency, consistency and cost-effectiveness of traditional hand-suturing without compromising the cosmetic or wound healing outcomes. These mechanisms and methods may automate the processes of tissue approximation, delivery of a needle and subsequent suture through the desired layer of tissue, as well as placing a stitch and detaching excess suture for interrupted stitches. The devices may be capable of performing any or all of the following actions, in either a mechanical or electrical fashion, whether triggered by the user or performed sequentially and triggered automatically:
(1) approximate tissue on opposing sides of an open wound by grasping each side of the wound independently or jointly,
(2) locking to secure the approximated tissue in the same location,
(3) inserting one or more needles through one or both sides of approximated tissue,
(4) securing the wound closed using a mechanism including, but not limited to, completing a knot-tying pattern using suture, a heat fusing method, or a mechanical locking mechanism,
(5) laying down the wound closure material (e.g. suture) onto the approximated tissue while maintaining tension, and
(6) removing any excess material that was not utilized by the wound closure mechanism (e.g. excess suture).

The devices may use suture or any similar tool to close any layer of tissue quickly and precisely. Any type of suture may be selected and/or loaded into the device to meet the necessary specifications for dissolvability, thickness, suture material type, etc. The apparatus may include a mechanism for loading of sutures prior to use and for removal after use. Delivery of interrupted or continuous suture may be selected intraoperatively. The device may also allow the user to select the type of suture material, gauge of suture, type of needle, the tension applied to each stitch, a buried or superficial closure, the number of knots and the length of the tail for interrupted sutures. The user may select the type of stitch, including, but not limited to, a simple superficial interrupted stitch, a simple buried interrupted stitch, an interrupted vertical or horizontal mattress stitch, a running/baseball stitch, a running subcuticular stitch, or a running horizontal mattress stitch, etc.

The devices may, in most cases, be utilized by a clinician to perform wound closure, but could, under certain circumstances, be utilized by any number of persons in any number of clinical and/or private settings.

The devices may be supplied pre-sterilized and/or disposable for single use, or re-sterilizable for multiple re-uses. The device may be sterilized pre-operatively to reduce risk of infection.

The devices may also have the capability to wirelessly connect via wireless communication protocols, e.g., Bluetooth®, Wifi, radiofrequency, or some other protocol in order to transmit information sensed, detected, or recorded by the system during use, to a remotely located server or controller. For example, the system may record the amount of tension placed on each stitch and wirelessly (or via wired connection or internal memory) transmit these data to a remote server or store it for later use. It may also have the capacity to measure the blood flow through tissue once the stitch has been delivered. Additionally, temperature sensors may enable the user to evaluate blood flow by measuring the heat signature local to the treatment site. Indicators may also be employed to indicate to the user vital metrics, such the remaining amount of suture in the device, the typical depth of each stitch, or the average tension applied to the stitches.

Adjustable instruments on either side of the device are applied to opposing sides of the desired layer of tissue to approximate the edges of the tissue to be closed. The user may control the delivery of a single stitch or a series of stitches at an adjustable interval to the desired layer of tissue. Sensors or guidance indicators may be used to ensure optimal and consistent closure. An instrument will perform the stitch or series of stitches as the operator guides the device along the tissue opening. This device may automate the processes of delivering the suture through the desired layer of tissue as well as knot tying and cutting the tail of the suture for interrupted stitches. Once the device has delivered the appropriate stitch(es), the user may remove excess suture material and release the tissue from the instruments on either side of the incision and the process may be repeated as necessary.

In one embodiment of the overall system, two separate surgical tools capable of communicating with one another through either a wired connection or wireless communication such as Bluetooth® to successfully pass the needle between them. The surgeon may use these ‘smart forceps’ that may be configured like, e.g., conventional tweezers, to pull the opposing sides of the tissue together with the appropriate amount of tension. When approximated appropriately, the surgeon may trigger the needle insertion that will be immediately followed by the device tying the square knot, then the device cuts the end of the suture. For continuous sutures, the insertion of the needle will be uncoupled with the knot tying process so that the surgeon can insert the needle in a spiral pattern at a chosen interval.

In a second embodiment of the overall system, the device may function similarly to a zippering mechanism, where the surgeon attaches the device to the tissue on opposing sides of the incision and the device is moved along the incision as the suture is delivered in a spiral pattern. Tractional wheels on either side of the device may continuously grasp tissue on either side of the incision and pull the tissue together to unify it prior to needle insertion. The needle is then inserted through both sides of tissue following the radius of the needle. A knot is tied inside the device and tightened down onto the tissue after needle insertion, then the device may cut the end of the suture. For continuous sutures, the insertion of the needle may be uncoupled with the knot tying process so that the surgeon can insert the needle in a spiral pattern at a chosen interval.

A third embodiment of the overall system involves a movable component on fused double forceps. This embodiment allows the forceps to draw together the opposing sides of tissue at the desired location along the incision. Once grasping the tissue in a desirable position, the forceps can be locked in place. The attachment on the forceps is a mechanical addition that delivers (a) stitch(es) and removes excess suture. For continuous sutures, the insertion of the needle will be uncoupled with the stitch delivery so that the surgeon can insert the needle in a circular pattern at a chosen interval.

Yet another embodiment involves a handheld device that initiates the automated, mechanical process of needle insertion, knot tying, and cutting with the squeeze of a trigger.

Any number of other overall system embodiments could encompass any combination of the iterations described below relating to the tasks of tissue approximation, holding the needle, inserting the needle through tissue, securing the approximated tissue in place, and removal of excess suture.

The following different iterations demonstrate how the device could approximate tissue. One embodiment involves a forcep device utilizing three grasping arms—a static arm, and a slideable or rotating arm on either side of the static arm, which forms 3 parallel arms to approximate tissue.

In another variation, the device may use optical sensors to automatically detect wound edges and approximate a given layer of tissue specified by the user.

In another variation, the user may approximate tissue using pre-existing surgical tools based on widely practiced methods prior to utilizing the wound closure device.

In another variation, tissue may be approximated by inserting rods along each edge of both sides of the incision. The rods may be brought together magnetically, or by creating a stitch around each rod. Once the stitches have been established, the rods may either be bioabsorbable and left in place, or may be removed by the user.

In yet another variation, tissue may be approximating by utilizing suction.

The following different iterations demonstrate how the device could hold the needle. The first iteration is a hinged clamping mechanism, the second iteration is a vice mechanism, and the third iteration is an aperture mechanism (similar to a camera shutter). In any iteration, the needle grasper may be, e.g., magnetic, to provide an additional layer of security so that the needle is not released accidentally. Moreover, these different embodiments are provided for illustrative purposes and are not intended to be limiting.

The following different iterations demonstrate how the device could insert the needle through approximated tissue on opposing sides of a wound. In one variation, the needle may be inserted into each side of the tissue independently (e.g., a first side and then a second side sequentially).

In another variation, the needle may be pushed through both sides of tissue, from the same direction, in one motion, circular or linear, after the tissue on either side of the incision is already opposed. The needle could be advanced by a lever that rotates in a semicircular fashion to advance the needle in a circular pattern through tissue.

In another variation, the needle may be advanced into the tissue using a circular guide which houses and guides the curved needle through a circular path. A lever which moves concentric to the circular guide is pushably apposed to the curved needle, such that actuation of the lever pushes the needle through the curved guide aperture, where it subsequently exits the guide aperture, and enters the tissue.

In another variation, the needle may be advanced into the tissue using a circular guide which houses and guides the curved needle through a circular path. A pair of soft, rotating capstan wheels are positioned such that they are tangent to the curved needle, and located on opposite sides of the needle. Rotation of the capstan wheels drive the needle through the curved guide aperture, where it subsequently exits the guide aperture, and enters the tissue.

In another variation, a straight, rather than curved needle, may be utilized. In this iteration, the needle is repeatedly inserted and removed from the tissue in a manner similar to a sewing machine. In this case, a needle drives a loop of stitching thread through fabric. As the needle reaches maximum travel through the fabric, a bobbin located underneath the fabric rotates and hooks the thread loop from the needle. The bobbin continues rotating, bringing the loop of thread completely underneath and around the bobbin. This motion causes the stitching thread to be looped around a secondary locking thread which is housed in the bobbin. The loop makes its way over the top of the bobbin, where the needle pulls the thread loop up through the fabric. The locking thread prevents the loop thread from coming out of the fabric.

The following different iterations demonstrate how the device could secure the approximated tissue on opposing sides of a wound. As the square knot is the knot most commonly utilized by surgeons for an interrupted suture closure, a corkscrew mechanism that the device may utilize to tie the square knot internally may be implemented. The suture may be passed through the center of a corkscrew pattern and held taught. The needle may then travel around the corkscrew pattern, e.g., in chirally opposite directions, in order to form the appropriate pattern to create a square knot. In between each wrap around the corkscrew rails, the knot may be tightened down by, e.g., mechanical arms.

Another iteration of a knot-tying mechanism involves the use of a pre-established pattern either manufactured into the device or a cartridge, or created by rotating features, used along with guide pins or grooves, to guide the suture ends into a pattern which enables the formation of a square knot.

One method to create a pre-established pattern of suture employs a plurality of static pins, a plurality of rotating pins, and a pattern of guide grooves and/or slots. In typical use, a first suture end is passed through a first guide slot, around a first static guide pin, and then hooked over a first rotating pin. This first rotating pin is then rotated a single turn clockwise, which imparts a single clockwise twist in the first suture end. The second suture end is then passed through the loop formed in the first suture end. Next, the second suture end is passed through a second guide slot, around a second static guide pin, and then hooked over a second rotating pin. The second rotating pin is then rotated a single turn counter-clockwise, which imparts a single counter-clockwise twist in the second suture end. The first suture end is then passed through the loop formed in the second suture end. The suture ends are then slipped out of the guide slots and off of the pins, and then pulled such that the knot is drawn down to the desired knot location.

An alternative to the square knot involves tying any number of other knots to secure the wound closure. Variations of the square knot include a “superficial” square knot (where the knot is tied closer to the surface of the wound), and a “buried or deep” square knot (where the knot is tied deep to the surface of the wound). Another alternative to the square knot involves tying any number of other knots to secure the wound closure, including, but not limited to a “Surgeon's” or “Friction” knot (where the suture is looped twice on the first throw and once on the second throw, as opposed to a square knot where the suture is looped once on both the first and second throws), or a “Granny” knot (where the first and second throws of the knot are in the same direction, as opposed to a square knot where the first and second throws of the knot are in opposite directions). Other knots options may include, but are not limited to the constrictor knot, clove hitch knot, modified surgeon's knot, single-double other side knot, strangle knot, modified miller's knot, etc.

Another alternative to the square knot is the use of heat, in order to thermally fuse two ends of a thermally fusible suture together, forming a closed suture stitch. The suture material can be standard suture material, such as polyester or nylon. The suture material can also be made of a bi-absorbable material such as PLA. In this concept, the two ends of suture which would typically be tied together using a knot, would instead be grasped by a pair of electronically heated forceps. The tips of the forceps employ electronic heating elements, such as but not limited to induction coils or radio-frequency heating elements.

The heated forceps are wired to a control unit which provides electrical energy to power the heating elements. The application of power/heat is controlled by the user, typically by depressing a button or switch. In typical use, the two suture ends are brought together, and held together at an intersecting location at the desired knot location. The ends are pulled such that the suture loop is under tension. The forceps are used to grasp the intersection of the two suture ends. Electrical power is applied, heating the forcep tips and melting the intersection of the two suture ends together, forming a closed suture stitch. When required, the fused suture stitch can be cut and removed from the patient. In the use of bio-absorbable suture material, the closed suture stitches will be organically absorbed by the patient over time.

Excess suture and other materials (e.g. the needle) may be removed from the site of wound closure by any number of methods involving tensioned suture and any combination of a sharp tooth around which the excess suture is wound, (a) mobile blade(s) that slices off the excess by moving in a linear pattern, or any number of other methods suitable for cutting suture.

### BRIEF DESCRIPTION OF THE DRAWINGS

FIG. 1 illustrates a perspective view of one embodiment of the system which may utilize two separate surgical tools used to facilitate suturing of a tissue region.

FIGS. 2A to 2C show side views of another embodiment of the device design utilizing a fused double forcep design.

FIG. 3 illustrates a side view of another embodiment of the system which utilizes a heated forceps tool to thermally melt and fuse two suture ends together, in lieu of a knot.

FIG. 4 illustrates a perspective view of another embodiment of the system which is a hand piece design for single hand use.

FIGS. 5A to 5F illustrate top, side, end, and perspective views of another embodiment of the system that utilizes fused double forceps to approximate tissue.

FIGS. 6A to 6C illustrate perspective and side views of the tissue approximation mechanism using the fused double forceps displayed in FIGS. 2A to 2C.

FIG. 7 illustrates a perspective view of another embodiment of the system which utilizes a set of three grasping arms which form three parallel arms to approximate tissue.

FIGS. 8A to 8C illustrates side and perspective views of three mechanisms which allow the device to grasp the needle.

FIGS. 9A to 9D illustrate end views of another embodiment which may utilize a motorized mechanism for facilitating passage of a needle through the tissue.

FIG. 9E illustrates a cross-sectional side view of the pattern that the needle may follow through the approximated tissue on either side of an incision.

FIG. 10 illustrates an end view of another embodiment of the system which may advance the needle into tissue using a rotating pusher knob.

FIG. 11 illustrates an end view of another embodiment of the system which may advance the needle into a pair of capstan wheels.

FIGS. 12A to 12D depict an example of how the suture device can be used to tie a square knot.

FIGS. 13A to 13G depicts another example by which the square knot could be tied inside the movable attachment described in FIG. 2.

FIG. 14 illustrates a schematic view of another embodiment of the system which may create a square knot by utilizing a pattern of static and rotating pins, guides and slots.

FIG. 15 demonstrates one example of a pattern for creating a knot utilizing static and rotating pins, guides, and slots.

FIG. 16 illustrates a side view of an iteration in which the tail end of the suture may be inserted through tissue and grasped by a component that moves in a linear fashion through the loops of the pattern, grasps the suture, and passes back through the loops.

FIG. 17 depicts another mechanism by which the square knot can be laid onto the tissue from within the movable attachment described in FIG. 2 and subsequently tightened.

FIGS. 18A and 18B illustrate perspective views of yet another embodiment where the system may project one or more indicators onto the tissue at locations at which the needle may be inserted and removed from the tissue on either side of the incision.

### DETAILED DESCRIPTION OF THE INVENTION

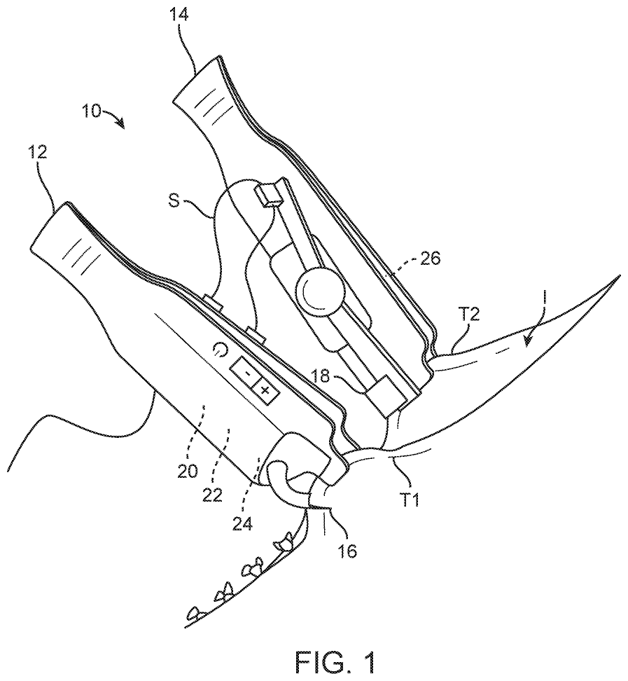

FIG. 1 illustrates a perspective view of one embodiment of the system 10 which may utilize two separate surgical tools 12, 14 similar to forceps which may be configured to communicate with one another through either a wired connection or wirelessly, e.g., Bluetooth®, infrared, radiofrequency, etc., to successfully pass the needle between them. The forceps have a mechanical knot tying mechanism attached to one or both of the tools 12, 14. The operator will use these ‘smart forceps’ to pull the opposing sides of the tissue T1, T2 together with the appropriate amount of tension. When approximated appropriately, the surgeon may trigger the needle 16 insertion that will be immediately followed by the device tying the square knot. The device then cuts the end of the suture tail. For continuous sutures, the insertion of the needle will be uncoupled with the knot tying process so that the surgeon can insert the needle 16 in a circular pattern at a chosen interval along the tissue opening. The first tool 12, for instance, may incorporate a radiused needle 16 which projects distally from the instrument while the second tool may incorporate a receiving channel 18 for receiving the needle which may have a length of suture S attached.

Each of the tools 12, 14 may be sized ergonomically for handling of each individual tool in each hand so that when in use, the practitioner may pass the needle 16 and attached suture length S between the tools while the system automates the passage of the needle and tensioning of the suture through the tissue. Each of the tools 12, 14 may be manipulated separately from one another by the practitioner while suture passage and tensioning is automatically provided by the tools 12, 14. Each of the tools 12, 14 may accordingly incorporate a processor or controller 20 and a transceiver 22 so that the tools may communicate with one another in coordinating the suturing and tensioning as well as recording of data and sensing of various parameters of the tool and/or tissue.

In use, the needle 16 may be passed entirely through the tissue from a first tool 12 to a second tool 14. In other variations, the needle 16 may instead be retained within the first tool 12 after being advanced at least partially through the tissue to pass the suture to the second tool 14.

The processor or controller 20 may be programmable to control a variety of parameters such as a preselected level of tension to be placed on the length of suture S in forming a stitch or select for delivery of an interrupted or continuous suture. The tools 12, 14 may also comprise one or more sensors 24, 26 for detecting or monitoring one or more parameters which may correspond to the tools themselves, or to the tissue regions to be sutured, or to both. For instance, the one or more parameters may include, e.g., a type of suture, a type of suture material, a type of needle, a type of tissue closure, a number of knots formed, a length of a tail for interrupted sutures, or a type of stitch selected.

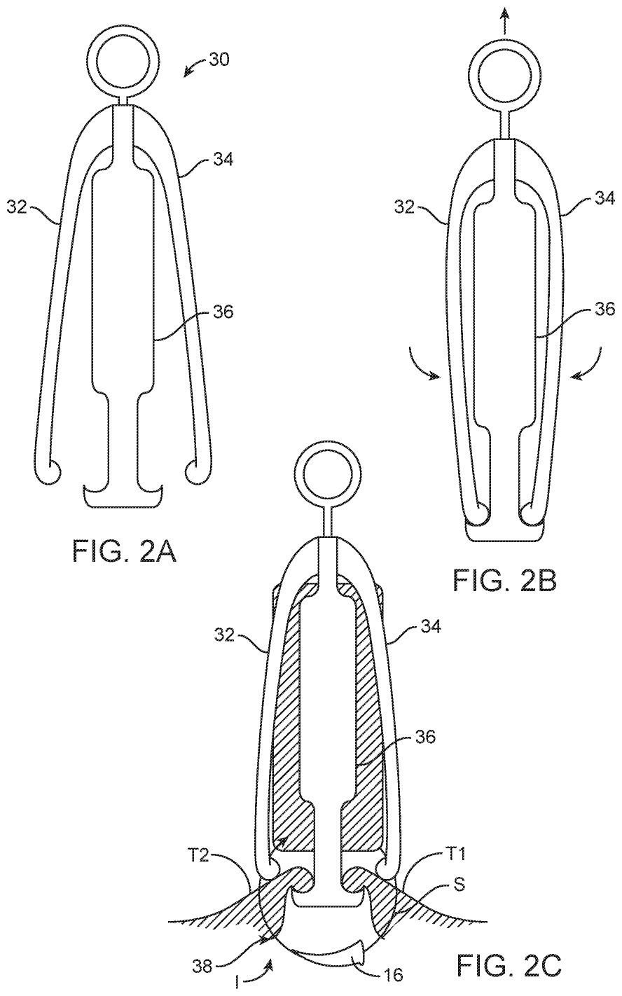

FIGS. 2A to 2C show side views of another embodiment of the device design 30 wherein a fused double forcep 32, 34 may have a movable attachment on the central shaft 36 of the forceps capable of delivering an interrupted or continuous suture through the approximated tissue.

FIG. 2A displays the forceps 30 in an unlocked position. This embodiment allows each component of the forcep 32, 34 to draw together the opposing sides of tissue T1, T2 at the desired location along the incision. Once grasping the tissue in a desirable position, the forceps 32, 34 can be locked in place. The process of locking the forceps 32, 34 may initiate the process of delivering a suture S. The attachment on the forceps delivers the needle 16 through the tissue, ties the knot, and cuts the end of the suture S. For continuous sutures, the insertion of the needle 16 may be uncoupled with the knot tying process so that the surgeon can insert the needle in a circular pattern 38 at a chosen interval.

FIG. 2B illustrates the fused double forceps in a locked position. In order to close the forceps, the operator may manually, automatically, mechanically, or electronically push together both outer limbs of the forceps 32, 34. In order to lock the forceps onto the tissue prior to needle delivery, one potential mechanism is to pull the central loop on top of the device up or away from the device to actuate the mechanism. Other potential mechanisms include applying more force to the closed forceps, pushing a button, flipping or sliding a switch, etc.

FIG. 2C demonstrates the fused double forceps grasping tissue T1, T2 on either side of an incision I in a locked position. It also shows the pathway of needle 16 delivery through the tissue from the attachment on the central shaft 36 of the fused double forceps 32, 34.

While each of the different embodiments are described herein, features from each of the embodiments such as the suture tying mechanisms and patterns described are intended to be used in any number of combinations with one another and are further intended to be within the scope of this disclosure.

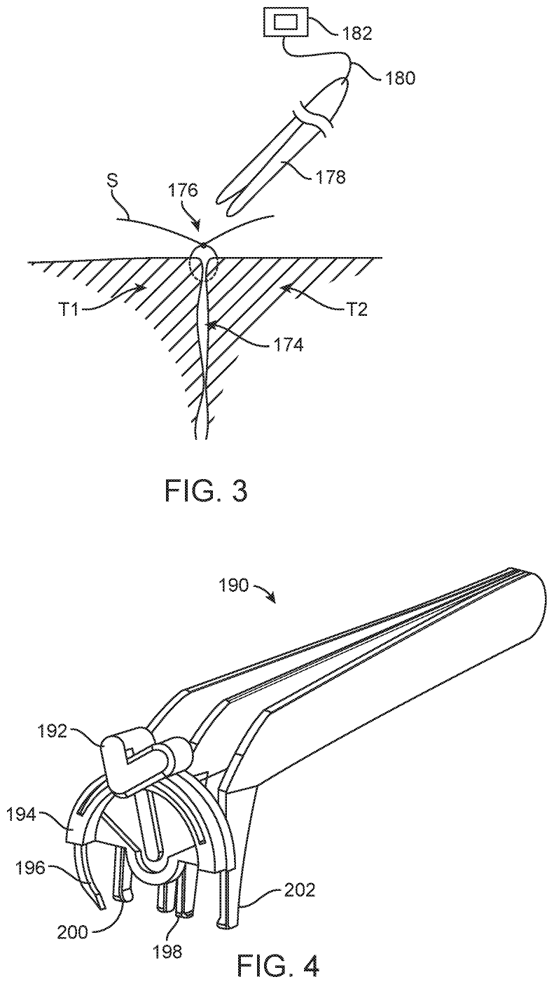

FIG. 3 illustrates a side view of another embodiment of the system which utilizes a heated forcep tool to thermally melt and fuse two suture ends together, in lieu of a knot. A suture S is stitched through tissue T1, T2, to close a wound 174. The ends of the suture may be intersected 176 or otherwise crossed upon one another and held together in position. A pair of heated forceps 178 may be advanced into proximity and closed upon the intersection 176 to pinch the suture. The forceps are connected by a cable 180 to a power generator 182. While the forceps 178 are pinched over the intersection 176, the forcep tips are heated by the power generator 182, until the heat fuses the two suture ends together at the intersection 176. The suture may be cut or released leaving the fused suture to maintain apposition of the tissue.

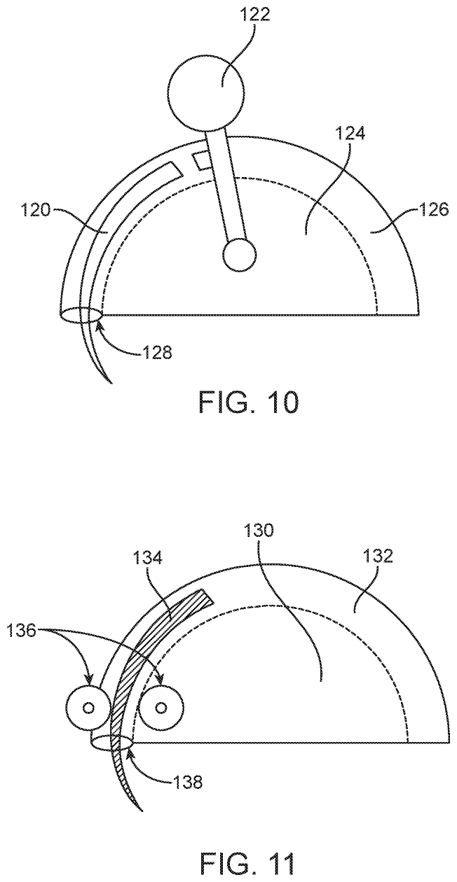

FIG. 4 illustrates a perspective view of an example of the system which is a hand piece design for single hand use that applies adjustable instruments on either side of the device to opposing sides of the desired layer of tissue to approximate the edges of the tissue to be closed. The system 190 may have two forceps arms which extend on opposed sides of a central forcep member. A first forcep arm may have a first grasping arm 200 and a second forcep arm may have a second grasping arm 202 extending on opposite sides of a central grasping arm 198 where each of the grasping arms 198, 200, 202 may extend an angle, e.g., transversely, relative to the forceps arms. A suturing assembly may be attached in proximity to the grasping arms such that the suturing assembly 194 may position a suture housing 194 into proximity of the grasping arms. The suture housing 194 may have an aperture for releasing a curved or arcuate needle 196 from within the suture housing 194 which may be manipulated via actuator 192 which may slide over the suture housing 194 to urge or force the needle 196 into and through the underlying tissue. The suture housing 194 may incorporate any of the mechanisms described herein such as those embodiments shown in FIGS. 10-11, 14-16.

In this embodiment, adjustable instruments on either side of the device may be applied to opposing sides of the desired layer of tissue to approximate the edges of the tissue to be closed. Tissue approximation could be performed by individual forceps or fused double forceps, as demonstrated in FIGS. 1-2. Either option allows individual maneuverability and grasping of each side of the wound. These instruments used to grasp tissue could mimic tweezers or could use any number of other mechanisms to grasp the tissue, such as suction, etc. If utilizing double forceps, the design will be such that each side of the forcep is able to be manipulated independently.

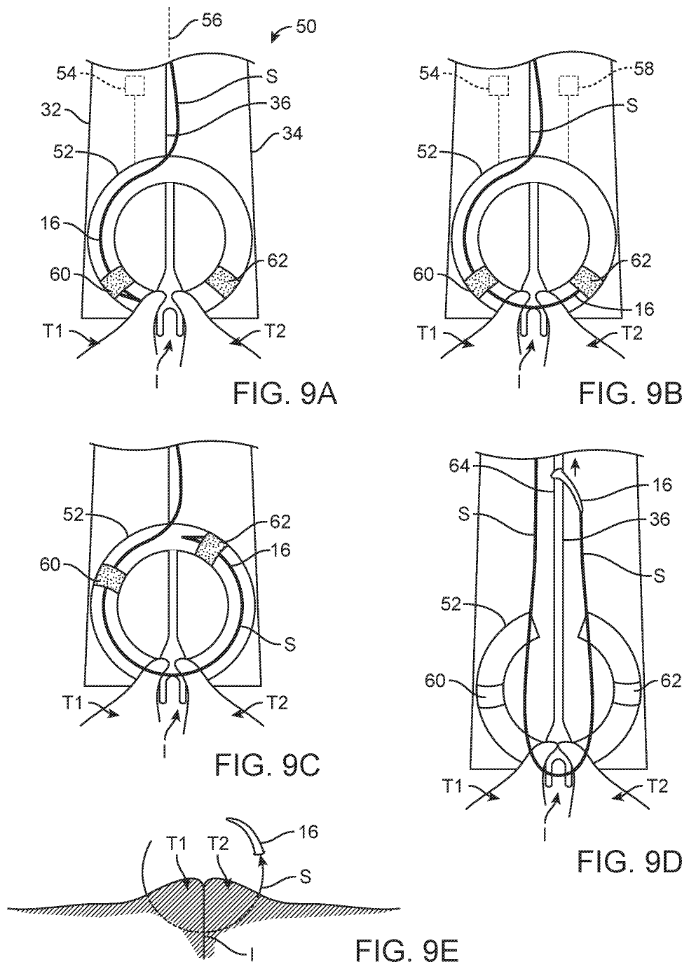

Once the tissue on either side of the wound has been grasped, the device could be locked in place by any number of mechanisms, including a mechanical switch, gears, etc. After the tissue is locked sturdily into the device, the operator may control the delivery of a single stitch or a series of stitches at an adjustable interval to the desired layer of tissue. The needle may be delivered through both sides of approximated tissue in a single circular pattern as demonstrated in FIG. 9E controlled by a lever pushing the butt of the needle as shown in FIG. 10 or advanced by another mechanism. Sensors or guidance indicators may be used to ensure optimal and consistent closure. The needle may be swadged or a pop-off. The suture would follow the circular pattern of the needle once inserted. After being inserted through the tissue, the needle could be removed from the suture or not. If removed from the suture, the needle could be disposed of in a cartridge attached to the device. Each needle and associated suture may be loaded into the device independently or via a cartridge carrying multiple needles.

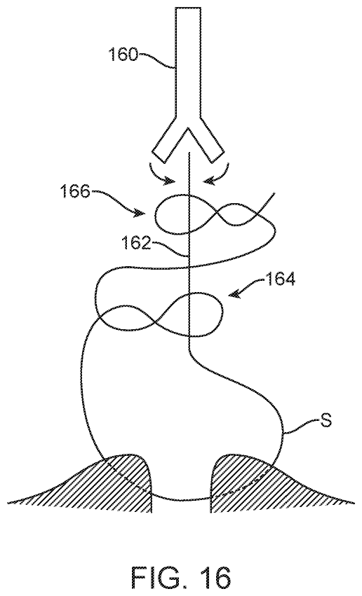

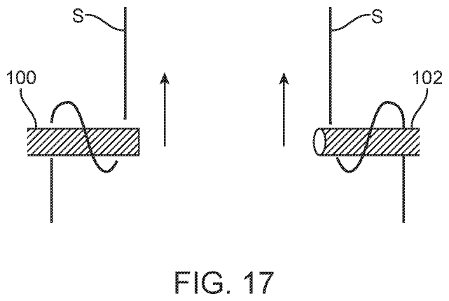

The pattern necessary to create a square knot, shown in FIG. 15, or the pattern to form another knot, may be pre-established and held inside the device, either within a cartridge or within the device itself. Additionally, a plurality of gears or gear-driven features are arranged into a pre-determined pattern to guide and advance suture material into a desired geometry. Once the needle has been passed through the approximated tissue, the recently inserted end of the suture may be fed through the pre-existing pattern to create a knot, as shown in FIG. 16. The device may maintain tension while mechanical arms lower the knot down onto the approximated tissue, as shown in FIG. 17. If the knot formed will be a square or surgeon's knot, each half-knot will need to be laid down independently of one another to maintain tension on the tissue. Any number of half-knots may be laid down onto the tissue based on the pre-established pattern (e.g. one iteration may have a pattern with two half-knots, as can be seen in FIG. 16, while another may have up to fourteen half-knots). The device may perform a stitch or series of stitches as the user guides the device along the tissue opening.

Once the knot has been laid onto the tissue, a blade, scissors, or any sharp object on a mobile arm may cut the tensioned, excess suture. Once the device has delivered the appropriate stitch(es) and the excess suture has been removed, the device operator may release the tissue from the instruments on either side of the incision and the process may be repeated as necessary.

The device may be operated by a single hand or two hands. All processes described herein may be performed mechanically or electrically, and each step may be manually triggered or automatically triggered by a preceding step. The actuator for each step or the entire process could be a button, trigger, switch, foot pedal, or any other number of actuation methods.

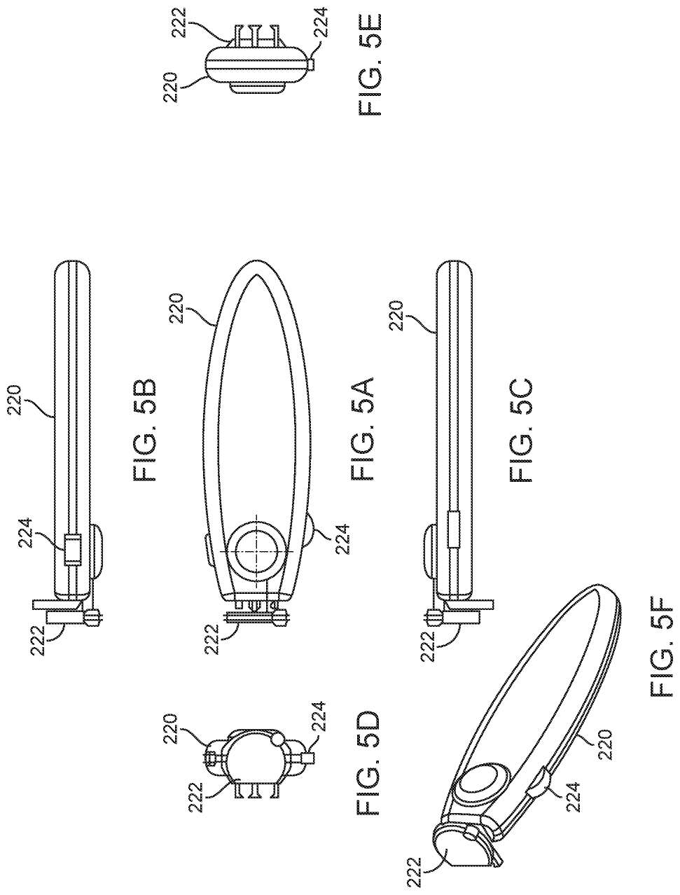

FIGS. 5A to 5F illustrate respective top, side, end, and perspective views of another embodiment of the system that utilizes fused double forceps to approximate tissue. This embodiment generally comprises a hand piece device, which can be operated and held using a single hand. The housing 220 of the device has an elongate shape, which can be held using the fingers, by wrapping the fingers around the circumference of the housing 220. This grip allows the thumb and index fingers of the same hand to operate various functions of the device, including but not limited to graspers, needle drivers, illuminators, sensors, and knot-tying mechanisms. The suture assembly 222 may be actuated via an actuator mechanism 224, e.g., positioned along a side portion of the housing 220, which may urge the needle and suture length through the underlying tissue and which may also form the knot through any of the mechanisms and patterns described herein.

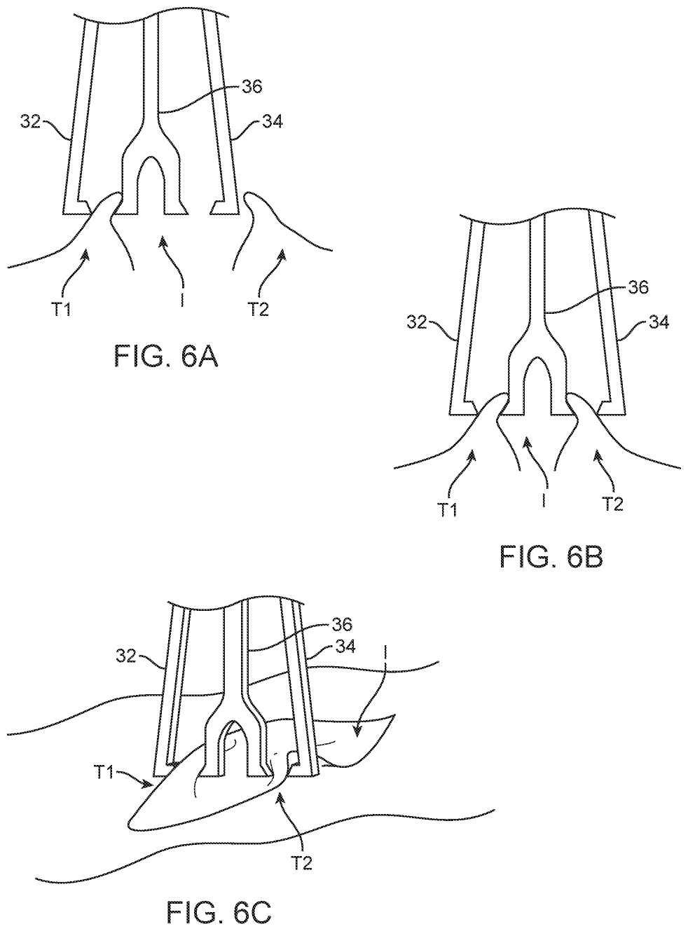

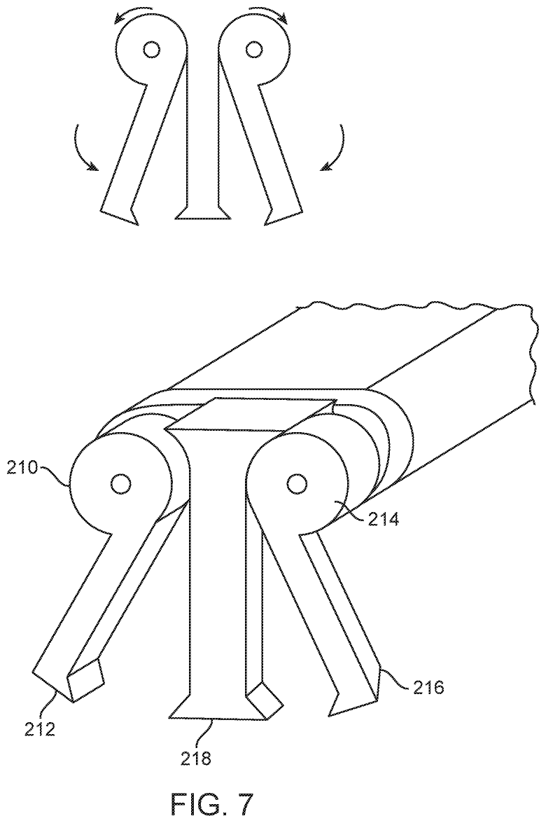

FIGS. 6A to 6C illustrate perspective and side views of the tissue approximation mechanism using the fused double forceps displayed in FIGS. 2A to 2C.

FIG. 6A displays the grasping of tissue on one side T1 of an open incision I.

FIG. 6B displays the grasp of a second tissue T2 on the opposing side of an open incision I.

FIG. 6C displays the use of the fused double forceps 32, 34 in order to pull opposing sides T1, T2 of an incision I together. Utilizing the fused double forceps 32, 34, the tissue from two sides of the incision can be pulled together (or, ‘approximated’) at the desired location along the incision by the operator.

FIG. 7 illustrates a perspective view of another embodiment of the system which utilizes a set of three grasping arms such as a static arm 218, and a slideable or rotating arm 212, 216 on either side of the static arm to approximate tissue. Each of the rotating arms 212, 216 may be pivoted or rotated about a corresponding pivot member 210, 214 which allow for the arms 212, 216 to rotate against the static arm 218, as indicated. The arms 212, 216 may be actuated simultaneously or separately from one another, as needed or desired, when manipulating the tissue to be approximated for suturing. This embodiment may also utilize any of the suture tying mechanisms and patterns described herein in order to tie the knot once the tissue has been approximated.

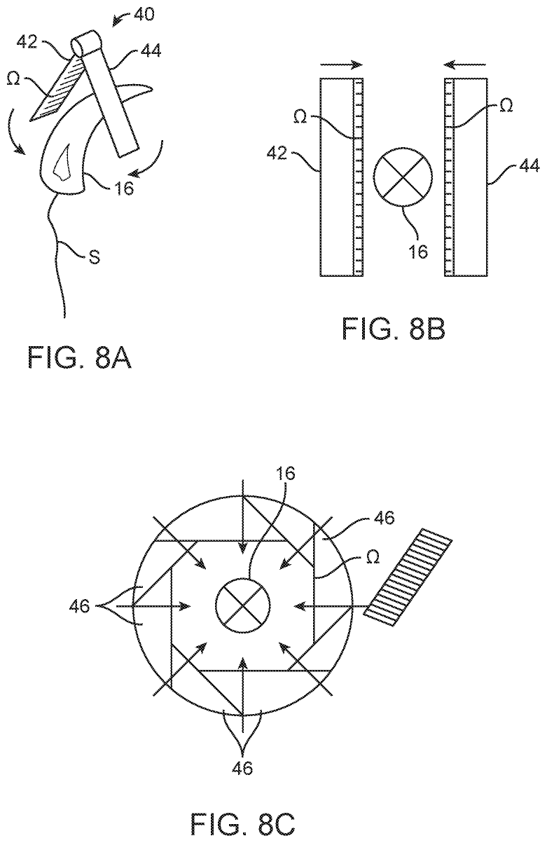

FIGS. 8A to 8C illustrates side and perspective views of three mechanisms which allow the device to grasp the needle. The omega (Ω) symbol indicates surfaces that can be made magnetic to enhance the capacity of the device to tightly grasp the magnetic surgical needles. Alternatively, the needle may be pushed along a specified trajectory without the use of any grasping mechanism.

FIG. 8A shows a perspective view of a hinged clamping mechanism 40. The hinge 40 may clamp down on the curved surgical needle 16 as shown.

FIG. 8B illustrates a top view of another embodiment comprised of a vice mechanism, where the needle 16 may be placed at the position of the ‘x’ between the two clamps 42, 44.

FIG. 8C illustrates a top view of an aperture mechanism 46 for grasping a needle 16 where the needle 16 may be placed at the position of the ‘x’ at the center of the aperture.

FIGS. 9A to 9D illustrate end views of another embodiment which may utilize a motorized mechanism for facilitating passage of a needle through the tissue.

FIG. 9A shows an end view of an elongate support structure 50 which incorporates the needle 16 coupled to a length of suture S which may be passed through an open channel 52 defined along a length of the structure. An actuation mechanism 54, e.g., a motor, may be incorporated within the structure, e.g., on one side of the tissue region to be sutured. The motor 54 may be operatively coupled to the needle 16 via a first receiving mechanism 60 and may drive the needle 16 in a curved or arcuate pathway through the structure and open channel 52 and through the tissue to be sutured, positioned within the open channel 52. The pathway of the needle 16 may traverse within a plane which is transversely defined relative to a longitudinal axis 56 of the structure to ensure that the needle 16 is inserted into the tissue, e.g., at a 90-degree angle relative to a surface of the tissue.

FIG. 9B shows the needle being inserted through the other side of the tissue and being actuated, e.g., by a second motor. The needle 16 may be pushed through the tissue T1 by the first motor 54. Once the needle tip appears on the other side of the tissue region T2, the second motor 58 or second receiving mechanism 62 may attach around it and ensure that the needle exits the tissue, e.g., at a 90-degree angle.

FIG. 9C shows the release of the needle from the first motor 54 and the second motor 58 pulls the suture S through the wound into the machine.

FIG. 9D depicts the needle 16 attaching to, e.g., the magnet 64, on the rod and being pulled into the device. A slit on the curved portion of the device will allow the suture S to exit the device once the tension is applied.

FIG. 9E shows a cross-sectional side view of the pattern that the needle 16 may follow through the approximated tissue on either side of an incision I.

FIG. 10 illustrates an end view of one embodiment of a needle insertion mechanism which may advance the needle 120 into tissue using a pusher knob 122. A needle housing 124 may house a curved needle 120 in a needle housing channel 126. The housing channel 126 may be curved or arcuate to accommodate the curvature of the needle 120 in a corresponding manner. Attached to the housing channel 126 is a movable actuating mechanism such as a knob 122 which may be actuated to maneuver in a plane aligned with the housing channel 126. The knob 122 may be coupled to a pushing mechanism within the housing channel 126 such that when the knob 122 is actuated, the pusher contained within the housing channel 126 may urge or force the needle 120 to advance through the housing channel 126 until it can exit via an aperture 128 for insertion into the underlying tissue to be treated.

FIG. 11 illustrates an end view of another embodiment of the system which may advance the needle 134 between a pair of capstan wheels 136. A curved or arcuate housing channel 130 may house curved needle 134. One or more actuators such as capstan wheels 136 may be positioned in proximity to the aperture 138 such that the wheels 136 are positioned to be tangent to the needle 134 on at least two opposite sides of the needle 134. When the capstan wheels 136 rotate, they advance the needle 134 through the channel 132, where the needle 134 can exit the housing via the aperture 138.

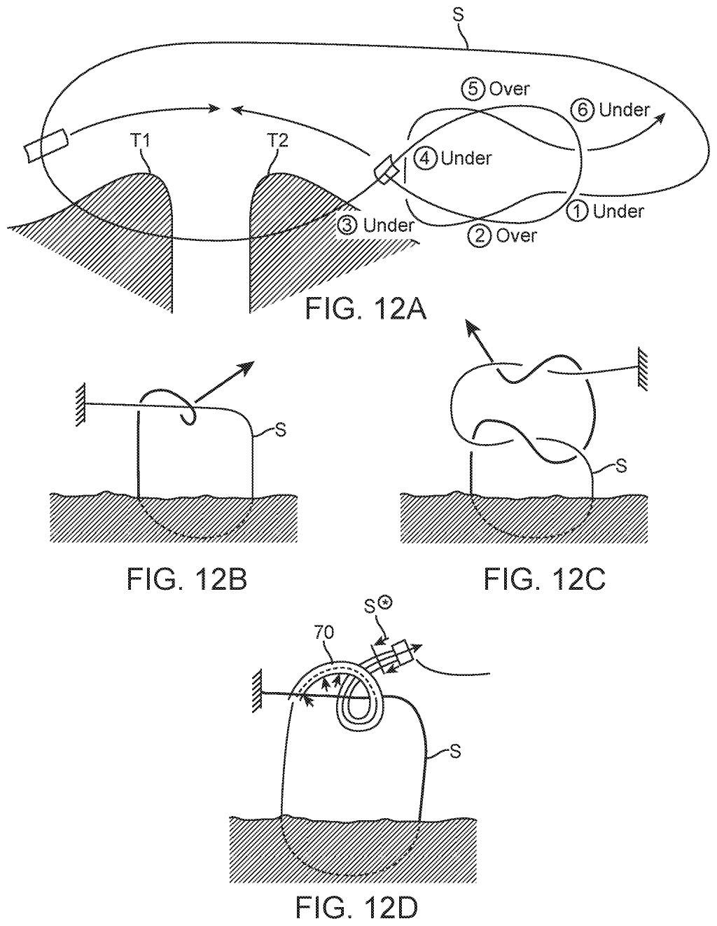

FIGS. 12A to 12D depict an example of how the device can be used to tie a square knot.

FIG. 12A shows the pattern that the suture S can follow in order to successfully form a square knot with only one end untethered.

FIG. 12B shows the initial ‘throw’ of the first part of a square knot with one end of the suture S held taught. The details depicted in FIG. 12B may be contained inside the movable attachment described in FIG. 2.

FIG. 12C shows the second ‘throw’ which forms the square knot pattern. The details depicted in FIG. 12B may be contained inside the movable attachment described in FIG. 2.

FIG. 12D depicts one embodiment of the mechanism by which each ‘throw’ of the square knot can be formed. By moving the needle 16 around in a specified pattern, e.g., on a corkscrew-like rail 70, with the fixed end of the suture S pulled taught through the center, the first throw can be formed. In order to form the second throw properly, a second corkscrew-like rail may be configured to be the chiral opposite.

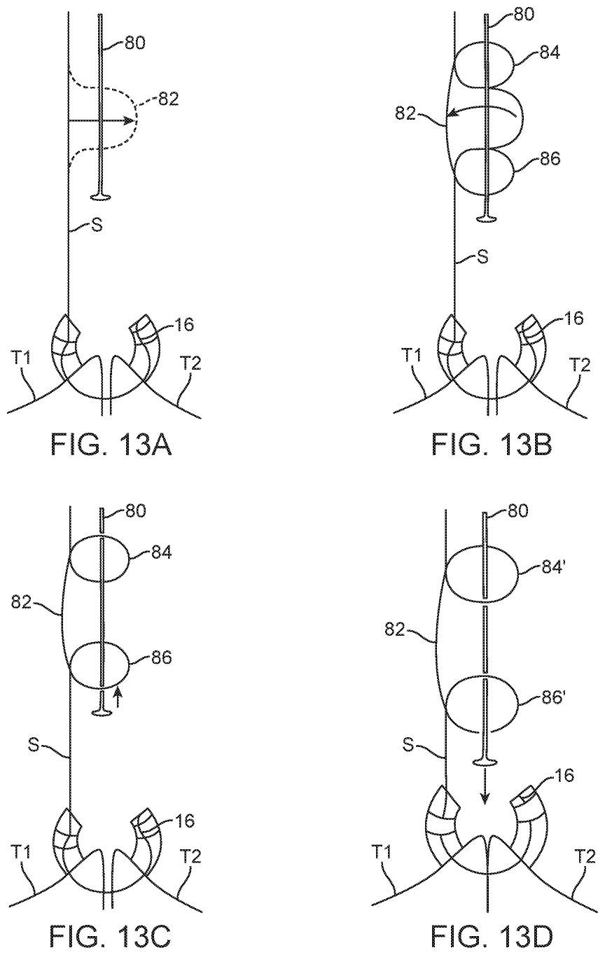

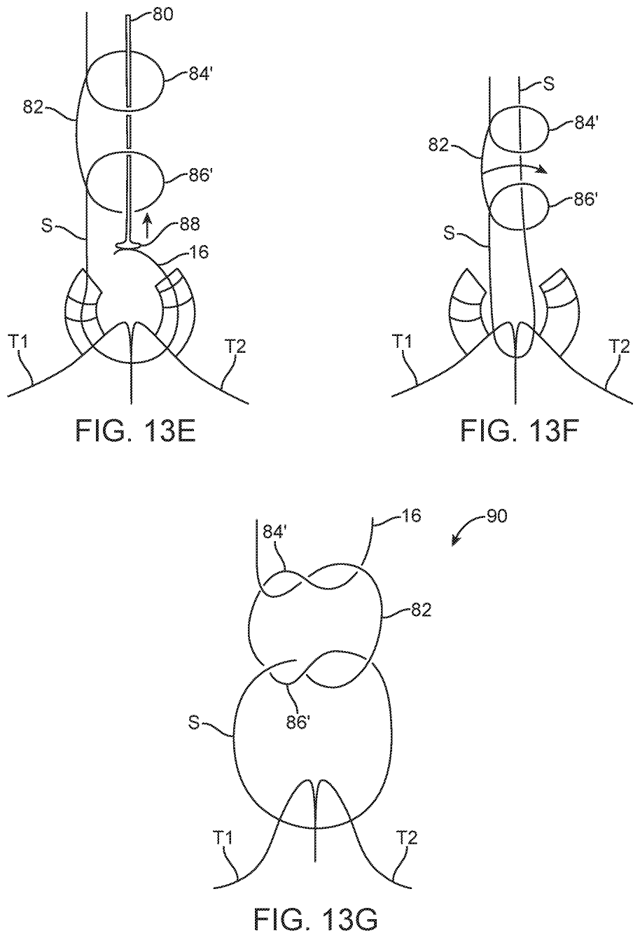

FIGS. 13A to 13G depicts another example by which the square knot could be tied inside the movable attachment described in FIG. 2. In one iteration, FIGS. 13A to 13F could be incorporated into a removable cartridge.

FIG. 13A shows a partial cross-sectional end view of the first step of the knot formation after the needle insertion has occurred. The suture S being supplied into the machine is looped 82 and pulled to the other side of a rod 80, which could be magnetic.

FIG. 13B shows the second step of the suture manipulation. The loop 82 in FIG. 7A is pulled all the way around the rod 80. As a result, two small loops of suture 84, 86 will be formed around the rod.

FIG. 13C depicts the movement of the rod 80 after the suture S has been manipulated from FIG. 13B. The rod 80 is pulled out of the suture configuration without disturbing the loops 84, 86 formed before.

FIG. 13D depicts the movement of the rod 80 back into the suture system.

Unlike before, the rod 80 may now be below the two middle strands and above the first and last strands of each respective loop 84′, 86′.

FIG. 13E depicts the attachment of the suture needle 16 to the grasping mechanism 88, e.g., magnet, at the end of the rod 80 and the rod's movement through the manipulated suture system from before. This may cause the square knot to be formed.

FIG. 13F shows the release of the knot from the system. The loop manipulated in FIG. 7B may now be released and a square knot may be formed.

FIG. 13G shows the resulting square knot 90.

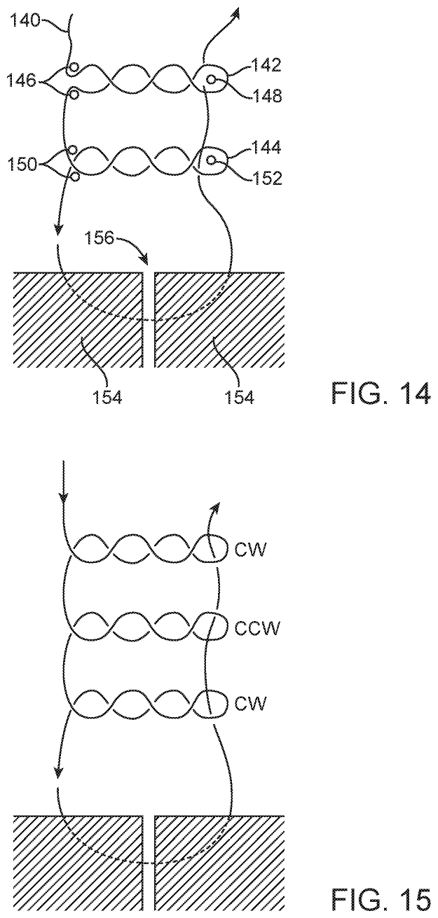

FIG. 14 illustrates a schematic view of another embodiment which may be implemented to create a square knot by utilizing a pattern of static and rotating pins, guides and slots. A length of suture 140 is loaded around a first pair of retractable guide pins 146 and also around a second pair of retractable guide pins 150 which are separate from one another. The first pair of guide pins 146 may be aligned linearly with the second pair of guide pins 150. These guide pins 146, 150 allow the suture 140 to be formed into a first twisted loop 142 in proximity to the first pair of guide pins 146. The first twisted loop 142 may be twisted at least once or several times to form one or more twists which are retained in position by a guide pin 148. The second twisted loop 144 in proximity to the second pair of guide pins 150 may similarly be twisted at least once or several times to form one or more loops which are retained in position by a guide pin 152. The first twisted loop 142 may be twisted in a first direction, while the second twisted loop 144 may be twisted in a second direction opposite from the first direction of twisted loop 142. The suture 140 is advanced through tissue 154, in order to close an incision 156. The suture 140 is passed through a terminal loop of the first twisted loop 144, and guide pins 150, 152 may be retracted to allow the suture S and first twisted loop 144 to collapse down to the tissue surface into a half knot. The suture 140 is then passed through a terminal loop of the second twisted loop 142, and then guide pins 146, 148 may be retracted to allow the suture S and loop to collapse down to the tissue surface into a second half knot, forming a full square knot. Although two apposed loops are shown, multiple loops may be formed in other variations.

FIG. 15 demonstrates one example of a pattern which may be created as a result of utilizing static and rotating pins, guides, and slots. This example illustrates a first twisted loop closest to the tissue surface having its loops twisted in a first direction, e.g., clockwise CW, with a second twisted loop twisted in the opposite direction from the first twisted loop, e.g., counter clockwise CCW. A third twisted loop may be incorporated such that the loops are twisted opposite to the second twisted loop, e.g., clockwise CW, as shown. Additional twisted loops may be utilized provided that they are twisted in alternating directions from adjacent loops.

FIG. 16 illustrates another embodiment in which a grasping mechanism 160 may be used to grasp and secure a tail end 162 of the suture S which has been inserted through tissue. The grasping mechanism 160 may be advanced linearly through the loops of the pattern and then retracted in a stepwise fashion, e.g., through first loop 164 and second adjacent loop 166, while grasping the suture tail end 162 to close the knot for tightening upon the tissue.

FIG. 17 depicts a mechanism by which the square knot can be laid onto the tissue from within the movable attachment described in FIG. 2 and subsequently tightened. The suture S may be wrapped around two rods 100, 102 inside the movable attachment, against which the suture S is pulled taught.

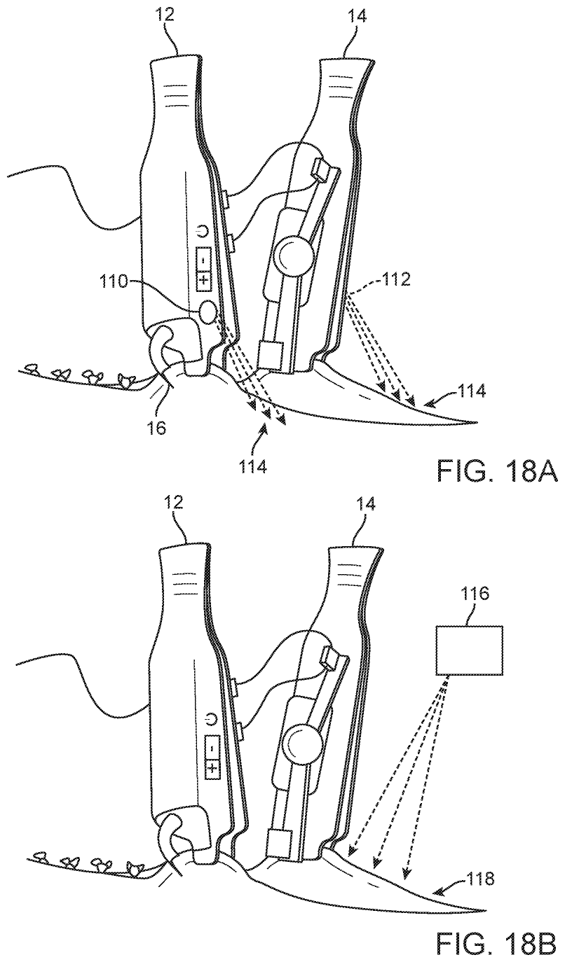

FIGS. 18A and 18B show perspective views of another embodiment in which the system may include a lighting mechanism for projecting one or more indicators onto the tissue at locations at which the needle may be inserted and removed from the tissue on either side of the incision.

FIG. 18A shows one variation where a light source 110, 112 (e.g., light emitting diode, laser, etc.) may be integrated with the system on one or both of the tools 12, 14. The light source 110, 112 may project at least one indicator 114 of where the needle 16 may be inserted next into the tissue or a plurality of indicators 114 may be projected in a pattern.

FIG. 18B shows another variation where a light source 116 may be removed from the instruments 12, 14 and positioned in proximity to the patient and tissue region to be sutured. The light source 116 may then project the one or more indicators or pattern 118 directly upon the tissue surface while remaining stationary.

While illustrative examples are described above, it will be apparent to one skilled in the art that various changes and modifications may be made therein. Moreover, various apparatus or procedures described above are also intended to be utilized in combination with one another, as practicable. The appended claims are intended to cover all such changes and modifications that fall within the true spirit and scope of the invention.

What is claimed is:

1. A tissue approximation system, comprising:
a first tissue grasper configured to grasp a first tissue region;
a second tissue grasper configured to grasp a second tissue region;
a needle having a length of suture coupled thereto and secured to the tissue grasper; and
a drive mechanism contained within at least the first tissue grasper, where actuation of the drive mechanism urges the needle to be advanced at least partially through the tissue held by each grasper when each of the graspers are in corresponding proximity to one another such that the length of suture is passed through the first and second tissue regions.

2. The system of claim 1 wherein the second tissue grasper is separate from the first tissue grasper.

3. The system of claim 2 wherein the first and second tissue graspers are sized for manipulation by two hands.

4. The system of claim 1 further comprising a securement mechanism configured to secure the length of suture.

5. The system of claim 1 further comprising a suction mechanism configured to maintain a hold on the tissue.

6. The system of claim 1 wherein the second tissue grasper is attached to the first tissue grasper.

7. The system of claim 1 wherein the first tissue grasper is sized for manipulation by a single hand.

8. The system of claim 1 wherein the first and/or second tissue grasper is sized for manipulation by a single hand.

9. The system of claim 1 wherein the first and second tissue graspers are configured to communicate with one another.

10. The system of claim 9 wherein the first and second tissue graspers are configured to communicate via wired or wireless communications.

11. The system of claim 1 wherein the system containing the first and/or second tissue graspers comprise a controller or processor.

12. The system of claim 11 wherein the controller or processor is programmable to control a preselected level of tension to be placed on the length of suture in forming a stitch.

13. The system of claim 11 wherein the controller or processor is programmable to select for delivery of an interrupted or continuous suture.

14. The system of claim 1 wherein the first and/or second tissue graspers comprise one or more sensors for detecting or monitoring one or more parameters.

15. The system of claim 14 wherein the one or more parameters correspond to the first and/or second tissue graspers.

16. The system of claim 14 wherein the one or more parameters correspond to the first and/or second tissue region.

17. The system of claim 14 wherein the one or more parameters are selected from the group consisting of type of suture, type of suture material, type of needle, type of tissue closure, number of knots formed, length of a tail for interrupted sutures, or type of stitch selected.

18. The system of claim 1 wherein the first and/or second tissue graspers comprise a memory component.

19. The system of claim 1 wherein the first and/or second tissue graspers comprise an indicator or alert.

20. The system of claim 1 wherein the first and/or second tissue graspers comprise a visual display.

21. The system of claim 1 wherein the first and/or second tissue graspers are configured to communicate with a remote processor.

22. The system of claim 1 wherein the needle is passed entirely through the tissue from the first tissue grasper to the second tissue grasper.

23. The system of claim 1 wherein the needle is retained within the first tissue grasper after being advanced at least partially through the tissue.

24. The system of claim 1 wherein the needle is advanced via the drive mechanism through a curved or arcuate pathway through the tissue.

25. The system of claim 1 wherein the needle is advanced via the drive mechanism through a linear pathway through the tissue.

26. The system of claim 1 wherein the needle is positioned relative to the first tissue grasper such that the needle enters a surface of the first tissue region at a 90 degree angle relative to the surface.

27. The system of claim 1 wherein the needle is positioned relative to the second tissue grasper such that the needle exits a surface of the second tissue region at a 90 degree angle relative to the surface.

28. The system of claim 1 wherein the drive mechanism is attachable to the first or second tissue grasper.

29. The system of claim 1 further comprising a component configured to project one or more indicators upon the first and/or second tissue regions for guiding insertion of the needle into the tissue.

30. The system of claim 29 wherein the indicator component is integrated with the first and/or second tissue grasper.

31. The system of claim 29 wherein the indicator component is positioned remotely from the first and/or second tissue grasper.

32. The system of claim 1 wherein the drive mechanism is configured to automatically controls tension of the length of suture.

33. The system of claim 1 further comprising a release mechanism for removing excess suture.

34. The system of claim 1 wherein the needle and length of suture are housed within a cartridge in communication with the actuation mechanism.

35. The system of claim 1 further comprising one or more sensors which are configured to detect a tension of the length of suture.

36. A method of approximating tissue, comprising:
securing a first region of tissue via a first tissue grasper;
securing a second region of tissue via a second tissue grasper separate from the first tissue grasper;
positioning the first and second tissue graspers into proximity with one another such that the first and second tissue regions are approximated towards one another; and
actuating a drive mechanism within at least the first tissue grasper such that a needle having a length of suture coupled thereto and secured to the first tissue grasper is urged from an initial point of needle release from the device and into corresponding receipt by the receiving point of the device whereby the length of suture is passed through the first and second tissue regions.

37. The method of claim 36 further comprising actuating a second drive mechanism within the second tissue grasper such that the needle is urged from the initial point of needle receipt back to the initial point of needle release whereby the length of suture is passed through the first and second tissue regions.

38. The method of claim 36 further comprising tightening the length of suture such that the first and second tissue regions are approximated towards one another.

39. The method of claim 38 further comprising automatically or manually controlling a tension of the length of suture.

40. The method of claim 38 further comprising recording a tension of the length of suture via the first and/or second tissue grasper.

41. The method of claim 36 further comprising recording of a depth of insertion within a wound.

42. The method of claim 41 further comprising detecting a blood flow within the first and second tissue regions.

43. The method of claim 36 further comprising tying the length of suture to maintain a position of the first and second tissue regions relative to one another.

44. The method of claim 36 further comprising fusing the length of suture with heat to maintain a position of the first and second tissue regions relative to one another.

45. The method of claim 36 further comprising securing the first and second tissue regions to one another.

46. The method of claim 45 further comprising removing excess suture from the first and second tissue regions.

47. A forceps tissue approximation system, comprising:
a central shaft;
a first forceps member coupled to the central shaft at a proximal end of the first forceps member and positioned to extend along the central shaft;
a second forceps member coupled to the central shaft at a proximal end of the second forceps member and positioned to extend along the central shaft in opposition to the first forceps member;
an actuation member configured to urge the first and second forceps members towards one another such that a position of the first and second forceps members are secured relative to one another;
a needle having a length of suture coupled thereto, wherein the needle is positioned within or along the central shaft and actuatable relative to the first and second forceps members; and
a securement mechanism configured to secure the length of suture.

48. The system of claim 47 further comprising a locking mechanism configured to lock a position of the first and second forceps members relative to one another.

49. The system of claim 47 wherein the securement mechanism is configured to form at least one knot with the length of suture.

50. The system of claim 47 wherein the securement mechanism maintains tension of the length of suture.

51. The system of claim 47 wherein the securement mechanism comprises a heating element.

52. The system of claim 47 further comprising a release mechanism for removing excess suture and other unnecessary materials from the site of wound closure.

53. The system of claim 47 wherein the needle and length of suture are housed within a cartridge in communication with the actuation mechanism.

54. The system of claim 47 further comprising one or more sensors configured to detect a tension of the securement mechanism.

55. The system of claim 47 further comprising one or more sensors configured to detect a blood flow within tissue.

56. A tissue approximation system, comprising:
an ergonomic support structure defining an open channel for receiving a region of tissue to be sutured to one another;
a needle having a length of suture attached thereto and secured within the support structure;
a first traction mechanism positioned along a first side of the support structure;
a second traction mechanism positioned along a second side of the support structure opposite to the first side;
an actuation mechanism configured to pass the needle from the first side to the second side of the support structure while the tissue region is positioned between the first and second traction mechanisms.

57. The system of claim 56 further comprising a locking mechanism configured to lock a position of a first and second forceps members relative to one another.

58. The system of claim 56 wherein the needle and length of suture are housed within a cartridge in communication with the actuation mechanism.
